(19) United States
(12) Patent Application Publication
(10) Pub. No.: US 2003/0143247 A1
(43) Pub. Date: Jul. 31, 2003

(54) Antiviral compositions and methods for identification and use

(76) Inventors: Hugh D. Robertson, New York, NY (US); Alita J. Lyons, New York, NY (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(21) Appl. No.: 10/355,455

(22) Filed: Jan. 30, 2003

Related U.S. Application Data
(60) Provisional application No. 60/353,071, filed on Jan. 30, 2002.

(51) Int. Cl. C12Q 1/68; C12P 19/34; A61K 39/12; C12N 7/00; C12N 7/01; C07K 2/00; C07K 4/00; C07K 5/00; C07K 7/00; C07K 14/00; C07K 16/00; C07K 17/00; A61K 38/00

(52) U.S. Cl. 424/204.1; 424/186.1; 435/6; 435/235.1; 530/300; 435/91.1

(57) ABSTRACT

A defined target for hepatitis and picornaviruses, and other viruses which utilize binding of the ribosomes to the internal ribosome entry site (“IRES”) of eukaryotic cells for viral protein translation, has been identified. Useful anti-viral compositions are compounds which block binding, physically or sterically, of the eukaryotic ribosome to the IRES to prevent translation of the viral proteins. The inhibitory molecule may bind to any ribosome subunit, initiation factor, or exposed site on the 40 S, 60 S, 48 S complex or 80 S complex in order to alter specificity of binding of the ribosome. The compounds are identified using the screening methods described herein from libraries of known compounds, including antibiotics. These compounds are then formulated for administration to a patient infected with or exposed to the virus.

### BACKGROUND OF THE INVENTION

This invention is generally in the field of methods of identifying, designing and using antiviral compounds, especially for treating infections with hepatitis or picornaviruses.

This application claims priority to U.S.S.N. 60/353,071 filed Jan. 30, 2002 “Antiviral Compositions and Methods for Identification and Use” by Hugh D. Robertson and Alita J. Lyons.

Many antibiotics in use today interfere with cell wall synthesis, protein synthesis, and nucleic acid synthesis of bacteria. Cell wall synthesis inhibitors, such as the beta-lactam antibiotics, ultimately lead to cell lysis due to the presence of tremendous osmotic pressure as a result of severe defects in the bacterial cell wall. Nucleic acid synthesis inhibitors, such as ciprofloxacin and sulphonamides, affect downstream processes such as protein synthesis and bacterial replication. Protein synthesis inhibitors, such as aminoglycosides, result in bacterial cell death, either because essential protein synthesis is halted or because the synthesis machinery is producing essential proteins that are dysfunctional. Many of the protein synthesis inhibitors directly affect the translational machinery of the bacterial cell.

A large number of antibiotics inhibit protein synthesis. The aminoglycosides are believed to attach to bacterial cell walls and enter the cells, where the aminoglycoside attaches to the cell's ribosomes and either shuts down translation, or alters it wherein the end result is the production of abnormal proteins. Because two-thirds of the ribosome's weight can be attributed to rRNA, it is not surprising that many protein synthesis inhibitors interact directly with rRNA. The size of the ribosome, as a whole, provides many targets from which to base screens for the identification of new and/or improved antibacterial agents. However, only a few ribosomal sites are utilized by antibiotics currently in use. For example, most of the clinically important antibiotics only interact with various motifs of the central loop of domain V of the large ribosomal subunit. Cundliffe, E., The Molecular Basis of Antibiotic Action, Gale et al., (Wiley, N.Y.), 1981, pp. 402-545; and Garrett, R. A. et al., Ribosomal RNA: Structure, Evolution, Processing, and Function in Protein Biosynthesis, Zimmerman, et al., (CRC, Boca Raton, Fla.), 1996, pp. 327-355.

Treatment of bacterial infections often involves the administration of more than one inhibitory compound. In combination with a first antibiotic compound, a second antibiotic may be administered to either increase the efficacy of the first compound or further damage the bacterial cell. For example, beta-lactams or vancomycin, may be administered in order to disrupt the cell wall in such a way as to allow the increased uptake of aminoglycosides or any other drug that functions intracellularly.

Many viruses rely upon the contribution of host cell protein components and energy for viability and replication. Host cell machinery such as the ribosomes, tRNA and various enzymes are required for the synthesis of the plethora of proteins required for virus replication.

One such virus that is dependent upon the host cell translational machinery is hepatitis C (HCV). HCV is an RNA virus. This virus, along with many others, mutates frequently, creating different genetic variations. These genetic variations provide the virus with a means to escape detection by the host immune system. Therefore, even if an immune system “catches up” with one particular form of the virus, the mutant strains that are present are then able to take over, undetected. These “camouflage” mechanisms used by certain viruses, and in particular HCV, make it extremely difficult for antibodies to elicit an appropriate immune response to rid the body of the virus. This relatively high rate of mutability is most likely related to the high propensity of inducing chronic infection (80%). Approximately 85% of the infected individuals, whether or not the disease progresses to a chronic stage, will harbor the virus for a lifetime.

Many of the current treatments revolve around interferon-α (IFN-α). It is believed that IFN-α binds to cellular receptors and initiates an intracellular response that includes enzymes involved in protein synthesis. This ultimately leads to the anti-viral activity/response. However, data from various clinical trials have shown that approximately 40% of patients treated with IFN-α initially responded to the therapy, but 70% of these relapsed after the treatment ended. Damen, M., and Bresters, D., in H. W. (ed.): Curr. Stud. Hematol. Blood Transf., Darger Publishers 1998, Basel. Overall, the long-term therapeutic effect and response was observed in only 10 to 30% of the patients. Houghton, M., in Fields, B. N. et al., Fields Virology, Raven Publishers 1996, Philadelphia. In addition many side effects were observed such as severe flu, fatigue, muscle and head aches, even depression, weight loss and diarrhea. Damen, M., and Bresters, D., in H. W. (ed.): Curr. Stud. Hematol. Blood Transf., Darger Publishers 1998, Basel.

An alternative treatment for HCV has been ribavirin. Ribavirin is an anti-viral with a broad range of target viral activities. Ribavirin is a guanosine analogue harboring a modified base (1-β-D-ribo-furanosyl-1,2,4-trizole-3-carboxamide), and has been proposed to inhibit the cellular enzyme inosine monophosphate dehydrogenase, resulting in a decrease of guanosine triphosphate. Damen, M., and Bresters, D., in H. W. (ed.): Curr. Stud. Hematol. Blood Transf., Darger Publishers 1998, Basel. However, ribavirin will cause side effects. Christie, J. M. and Chapman, R. W., Hosp Med. 60, 357 (1999). In particular ribavirin accumulates in the erythrocytes of patients and can cause hemolytic anemia.

It is therefore an object of the present invention to provide a method to screen compounds for their ability to selectively inhibit IRES-dependent protein synthesis in virus-infected mammalian cells, and compounds identified using this method.

It is a further object of the present invention to provide methods and compositions for use in treating hepatitis and picornavirus infections, especially those caused by hepatitis C.

### BRIEF SUMMARY OF THE INVENTION

A defined target for hepatitis and picornaviruses, and other viruses which utilize binding of the ribosomes to the internal ribosome entry site (“IRES”) of eukaryotic cells for viral protein translation, has been identified. The detailed structure analysis of eukaryotic and prokaryotic ribosomes, their well understood mechanisms of action in protein translation, and what is known in the art regarding the mechanisms of antibacterial compounds, provide a detailed framework from which to use methods to screen for therapeutic compounds for the treatment of humans or animals infected with a virus dependent upon IRES-mediated translation.

Useful anti-viral compositions are compounds which block binding, physically or sterically, of the eukaryotic ribosome to the IRES to prevent translation of the viral proteins. The inhibitory molecule may bind to any ribosome subunit, initiation factor, or exposed site on the 40 S, 60 S, 48 S complex or 80 S complex in order to alter specificity of binding of the ribosome. The compounds are identified using the screening methods described herein from libraries of known compounds, including antibiotics. Although in some cases the compounds will be known for use, for example, as antibiotics, various formulations will be screened to provide an effective dosage for antiviral applications. These compounds are then formulated for administration to a patient infected with or exposed to the virus. An effective amount is administered to selectively inhibit internally initiated translation of an mRNA by ribosomes utilizing the IRES-mediated mechanism of access to the mRNA, but not inhibiting cap-dependent translation. The compounds can be administered alone or in combination with other antiviral, antibiotic, antifungal, or anti-inflammatory compounds.

### BRIEF DESCRIPTION OF THE DRAWINGS

FIG. 1 is a diagram showing the translation initiation pathway of the HCV IRES, indicating the sites where the compounds bind to the ribosome to selectively block IRES-mediated translation.

### DETAILED DESCRIPTION OF THE INVENTION

Protein Production in Eukaryotes, Prokaryotes and Viruses.

Proteins comprise more than half of the total dry mass of a cell. Proteins are the machinery that drive growth, development, and maintenance of cells and viruses. Therefore, an understanding of protein synthesis is critical to understanding the basic mechanisms that drive molecular activity central to cell viability and virus replication. The process of protein synthesis is called “translation”.

Ribosomes read the genetic message of the mRNA and translate the message into protein. Ribosomes are conglomerates of proteins and RNA (rRNA). Eukaryotic and prokaryotic ribosomes are composed of a large and a small subunit that come together to form a large complex of several million daltons. The small subunit binds the mRNA and the tRNAs, while the large subunit catalyzes peptide bond formation.

The components of the ribosomes are defined by their “S” value (Svedberg unit), which defines the rate of sedimentation in a centrifuge. The small ribosomal subunits (30S in prokaryotes and 40S in eukaryotes) have a head and a base with an armlike platform extension. The small eukaryotic ribosome subunit also harbors another extension from the head and lobes that is believed to contain additional rRNA sequences. The large subunit (50S in prokaryotes and 60S in eukaryotes) has features that are referred to as the protuberance, stalk and the ridge. The large subunit also harbors a channel or tunnel in which the assembled polypeptide chain will exit the ribosome. Four different ribosomal RNA molecules (rRNA) are part of each eukaryotic ribosome: 18 S, 5.8 S, 28 S, and 5 S rRNA. The rRNA molecules are critical in protein synthesis. The 28 S subunit participates in the peptidyl transferase activity of the 60 S subunit. The 18 S subunit rRNA aids in positioning the mRNA with the correct peptidyl tRNA molecule. All rRNAs contribute structural characteristics to the overall ribosome. The rRNAs are responsible for providing the structural framework onto which the ribosomal proteins assemble.

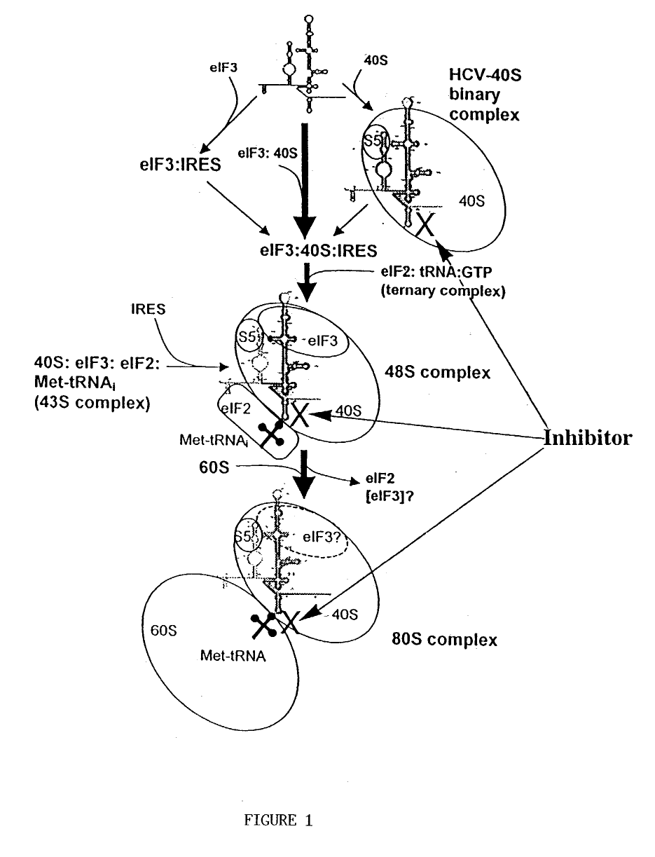

Eukaryotic initiation factors (eIF) mediate the assembly of the 80 S ribosome. As partially depicted in FIG. 1, the process driving 80 S formation can be subdivided into different stages of development:

1) 43 S pre-initiation complex is formed by the addition of a eIF2/initiator tRNA (Met-tRNAᵢ)/GTP ternary complex and other eIFs to the 40 S subunit.

2) The 43 S complex binds to the mRNA. In general, eIF4E (the cap-binding factor) subunit of eIF4F initially recognizes the 5′ G-cap. However, as will be further discussed below, binding may be cap-independent. In cells infected with the hepatitis C virus (HCV), for example, initiation factors, including eIF2 and eIF3, bind to the initiation codon as a result of the 40 S subunit interacting with IRES elements of the HCV virus and promote 80 S assembly at the IRES element. The 40S subunit binds to the virus RNA independent of all initiation factors. Pestova et al., Genes Dev., 12, 67-83, 1998; and Pestova, T. V. and Helen, C. U. T, Virology, 258, 249-256, 1999.

3) The 43 S complex scans the 5′ untranslated region (5′ UTR) from the 5′ cap to the initiation codon. At the initiation codon the 43 S complex is converted to a 48 S initiation complex in which the initiator tRNA is paired with the initiation codon.

4) The 48 S complex is displaced and joined with the 60 S subunit to form the 80 S ribosome. This formation of the 80 S ribosome is dependent upon the eIF5B, which harbors an essential ribosome-dependent GTPase activity and this displacement leaves Met-tRNAᵢ in the ribosomal P site. Pestova, T. V., et al. Proc. Nat'l Acad. Sci., 98(13), 7029-7036, 2001.

Generally, ribosomes initiate translation of eukaryotic mRNAs at an AUG codon downstream of a mRNA 5′ cap structure (7-methylguanosine linked to a triphosphate). After binding the 5′ cap, the ribosome scans the mRNA for the proper AUG codon, usually the first one encountered.

Eukaryotic ribosomes recognize termination signals and dissociate very rapidly from the mRNA. This dissociation prevents the reinitiation of translation at a downstream AUG. Therefore, the ribosome scanning mechanism from the 5′ cap and the inability to reinitiate at internal AUGs largely results in single proteins that begin with a methionine residue encoded by the first AUG that the scanning ribosome recognizes. As will be discussed below, mammalian ribosomes can use a second pathway to translate non-capped mRNAs. This ability can be damaging if the host cell ribosomes become dedicated to another translational process that is involved in generating protein detrimental to the well-being of the cell, such as that encoded by a virus.

It was previously thought that the mechanism of protein synthesis was the same for bacteria and the viruses which grow on them, and that another mechanism of protein synthesis was common to eukaryotic cells and the viruses which they support. This is true in general. However, the existence of important exceptions to these ideas—in particular that there exists a class of viruses which grow in eukaryotic cells and which use a prokaryotic-like mechanism of translation initiation—provides the framework for the methods and antivirals described herein. The study of Legon, et al. showed that mammalian ribosomes can recognize and bind to internal ribosome binding sites present on viral mRNAs isolated from bacteria, and explicitly cited the similarity of this process to the binding of eukaryotic ribosomes to an IRES-bearing picornaviral mRNA. Legon, et al., PNAS, USA, 74, 2692-2696. Bacterial ribosomes, on the contrary, are unable to recognize mammalian mRNAs containing “caps”, thus ruling out an external ribosome binding mechanism for ribosomes in bacteria. For 20 years, no one has tried to block translation at IRES sites.

The IRES elements of viruses are sequences of mRNA that allow the protein synthesis machinery of the host cell to make viral proteins in addition to making cellular proteins. IRESs are defined solely on the basis of functionality; not by sequence or structural motifs. IRES elements can function where there is a shut down of cap-dependent host protein synthesis, while still allowing for efficient translation of viral mRNA. Cryo-electron microscopy (Cryo-EM) has been used to create high-resolution three dimensional maps of proteins, protein complexes, and protein nucleic acid complexes. Cryo-EM allowed researchers to visualize ribosomes binding to wild-type IRES elements. When three dimensional maps are used to visualize truncated and wild-type IRES elements that are bound and unbound by ribosomes, one observes conformational changes that are induced in the ribosome by wild-type IRES elements. Spahn et al., Science, 291(5510), 1959-1962. This induced conformational change may allow the ribosome to “clamp down” onto the mRNA to produce efficient initiation of viral protein production. Such IRES induced structural changes in the ribosome may also help to explain how one set of ribosomes can carry out two mechanistically different kinds of protein translation.

Many anti-viral therapies and current research geared towards the elucidation of potential inhibitors of IRES mediated translation rely upon mRNA mimicking, IRES mimicking, or protein mimicking formulations. These formulations or compounds typically serve to titrate out the required or essential components thought to be required for internal initiation of translation. However, many of the very same components are required for efficient and normal cellular translation. Therefore, it is not a foregone conclusion that the titration of cellular components required for internal translation will not interfere or disrupt translation.

Prokaryotic and many viral mRNAs do not contain 5′ caps. They do, however, utilize internal ribosome entry sites (“IRES” or “IRES elements”). Such elements provide the cell or virus with a mechanism to produce more than one protein from a single mRNA transcript. It is these IRES elements that have been found to be particularly sensitive targets for inhibition of translation of certain viruses, especially hepatitis viruses, most especially hepatitis C, and picornaviruses such as the virus that causes hoof and mouth disease.

Some work has been published over the past few years concerning the drug-binding ability of certain highly structured RNA molecules. One class of compounds which binds RNA is the aminoglycosides, including the gentamicin, kanamycin and neomycin groups. Members of this chemical family of compounds can bind RNA and interfere with activity, as measured by in vitro assays using purified synthetic RNA molecules. For example, von Ahsen, U., et al. (Nature 353, 368-370, 1991) show that several aminoglycosides block ribozyme-catalyzed in vitro RNA splicing in group I introns encoded by phage T4. Zapp, M. L., et al. (Cell 74, 969-978, 1993) show that neomycin B binds HIV viral RNA region within the Rev-binding site and blocks Rev protein binding. Chen, Q., et al. (Biochemistry 36, 11402-11407, 1997) have found that three aminoglycosides exhibited pronounced stabilization of RNA duplexes against thermal denaturation. See also “Translation of hepatitis C virus RNA” (Hellen, C. U. T. & Pestova, T. V., J. Viral Hepatitis 6, 79-87 [1999].) As described herein, this use of aminoglycosides involves binding to free RNA molecules containing highly structured regions. The compounds screened are chosen from those likely to inhibit the ribosome's ability to translate HCV IRES-bearing mRNAs while continuing translation of host-cell capped mRNAs.

I. Methods of Identifying Antiviral Compounds

Antiviral compounds binding to ribosome subunits, initiation factors or an exposed site on an 80 S ribosome site can be identified using the following methods.

FIG. 1 indicates the sites against which the desired antiviral compounds are targeted and outlines the steps in the translation initiation pathway used by the HCV IRES, as well as an example of how to inhibit this process selectively. As discussed above, the start of HCV protein synthesis, as well as that stimulated by other IRES-based mRNAs, does not necessarily use all of the same initiation factors or ribosomal sites as does eukaryotic protein synthesis stimulated by capped mRNAs. Also, the order in which such factors and ribosomal sites bind to the IRES appears to differ from that of capped mRNAs. Specifically, IRES rebosome interaction has a number of features in common with prokaryotic ribosome binding, even though it occurs in a eukaryotic cell, Thus, it is likedly that there are a number of sites on the eukaryotic ribosome which are used exclusively for the IRES-directed pathway, and which can therefore serve as targets for selective inhibition of IRES-based translation.

The upper part of FIG. 1 shows that the favored initial reaction between the HCV IRES and the translation machinery occurs with the 40 S ribosomal subunit, yielding a binary complex. The RNA domains of the HCV IRES on the 40 S subunit are highlighted, showing that at this stage, both stem-loop II (shown bound to ribosomal protein S5) and stem-loop III are involved in the binding reaction. Subsequently, eukaryotic initiation factor 3 (eIF3) and the ternary complex of eIF2:Met-tRNA1:GTP are added stepwise, leading to the formation of the 48 S complex shown in the middle of FIG. 1. At this stage, the HCV IRES shifts in structure so that only stem-loop III (highlighted) is bound to the 40 S subunit and now to eIF3 as well. At this stage, the initiator Met-tRNA (black “cloverleaf”) and eIF2 are adjacent to the part of the IRES containing the AUG initiator triplet.

Addition of the 60 S ribosomal subunit leads to the formation of the 80 S complex depicted in the lower part of FIG. 1. Now additional sequences of the HCV IRES (highlighted) downstream from the initiator AUG (still adjacent to initiator Met-tRNA) are involved in complex formation. In traditional 80 S initiation complexes with capped mRNAs, no initiation factors remain. However, HCV protein synthesis may involve the use of noncanonical factors or of canonical factors in a noncanonical manner. It has been suggested, for example, that eIF3 remains bound to the 80S ribosomal complex, as shown in the lower part of FIG. 1.

All three of the HCV IRES:ribosomal complexes depicted here are subject to selective inhibition. Because of the unique manner in which the HCV IRES forms a binary complex with the 40 S ribosomal subunit, the most likely place for an inhibitor to bind is shown as a site on the 40 S subunit which is selectively required for stable IRES binding (marked by “X”). The inhibitor is also shown in FIG. 1 as inhibiting the 48 S and 80 S complexes, since the inhibitor could halt the ribosome binding process at any of these three levels. In addition, additional binding sites for selective inhibitors of IRES-based protein synthesis are expected to be present and accessible on the ribosomal particles.

Selective inhibition of translation may be demonstrated by in vitro methods well established in the art. For example, the molecular mechanisms involved in eukaryotic translation have been exploited through the use of purified ribosomal components, translation initiation and elongation factors, and recombinant RNA constructs comprising both IRES- and 5′ cap-mediated ribosome entry sites. Pestova, T. V. et al., Proc. Natl. Acad. Sci., (98)13, 7029-7036, 2001; and U.S. Pat. Nos. 5,989,904 and 6,291,637. Additional rRNA footprinting assays reveal the site(s) on the ribosome to which the compound binds and inhibits. Assays such as footprinting, incorporating primer extension analyses, and S1 nuclease protection are well known within the art.

Cell extracts or lysates may also be used to recapitulate the translation process and determine the effect(s) of the inhibitory compound. For example, rabbit reticulocyte lysates and HeLa cell extracts have been used extensively in the art to elucidate those protein components required for internal initiation of translation. Brown, B. A. et al., Virology, 97:376-405, (1979); and Dorner, H. A. et al., J. Virol., 50:507-514, (1984). Yeast cells and yeast cell lysates may also be incorporated into an overall scheme to determine the efficacy of the inhibitory compound. It is well known that yeast cells are incapable of translating poliovirus RNA, in vivo and in vitro. This inhibitory effect is dependent upon the 5′ untranslated region (UTR) of the viral RNA and a trans-acting RNA factor. Coward, P. et al. J. Virol., 66:286-295, (1992). Thus, the yeast system provides a useful model to genetically alter the properties of the trans-acting RNA factor and determine the effects of the inhibitory compound. Alternatively, cell extracts may be depleted of proteins that may interfere with assessing the efficacy of the compound to be studied. For example, immuno-depleting extracts via monoclonal or polyclonal antibodies is a well-established method.

One of skill in the art will realize that many additional methods exist for identifying compounds that attenuate or abolish the interaction between an inhibitory compound and, for example, the target ribosome subunit or initiation factor. For example, one may incorporate a two-hybrid system to detect protein/protein interaction. One may transform or transfect the appropriate host cell with a DNA construct comprising a regulatable promoter controlling a reporter gene of interest. The promoter is regulated by a transcription factor having a DNA binding domain and an activating domain. The domains are separated and genetically fused to the inhibitory compound of interest and the initiation factor or ribosome subunit of interest (wherein the subunit may be either protein or rRNA). One hybrid will harbor the translation factor or inhibitory compound fused to the DNA binding domain and the other hybrid will harbor the transcription factor or inhibitory compound, not incorporated into the first hybrid, fused to the activation domain. Providing the appropriate controls, familiar to one of skill in the art, will allow one to assess the inhibitory compound's ability to bind to the target translation factor based upon the output activity of the reporter gene.

In vitro translation assays may be utilized to assess the effect of the potential inhibitor on translation. In vitro translation assays may be used before or after information is gathered regarding the actual binding of the compound to a component of the translation machinery. To determine whether, or not, the compounds are capable of inhibiting internal initiation of translation, DNA constructs harboring two or more reporter genes are assayed for activity of each protein encoded by the construct. For example, a capped bicistronic construct containing the β-galactosidase and luciferase genes, wherein the 3′-most gene is flanked by a 5′ element representative of an IRES, may be used. Cap-independent translation will result in the expression and activity of the 3′-most gene (the internal gene), whereas cap-dependent translation will produce the reporter protein that is 5′ to the internal gene. For example, translation from the bicistronic message in an uninfected HeLa extract, will result in reporter activity for β-galactosidase and luciferase. Addition of an inhibitor specific for IRES-based translation will result in the expression of the 5′-most reporter gene.

Recent work has provided data that HCV undergoes genetic mutational changes while being cultured in hepatocytes and other cell lines, indicating virus replication in vitro. Rumin, S. et al., J. Gen. Virology, 80, 3007 (1999); Kato, N., et al., Jpn. J. Cancer Res. 87, 787 (1996). Further in vitro work has suggested that sufficient levels of viral RNA and protein are synthesized to levels detectable by Northern blots and radioisotopic labeling, respectively, indicating that in vitro work with regards to HCV is indeed feasible. Lohmann, V., et al., Science, 285, 110, (1999).

These in vitro assays may be subject to modification. The premise of the assays is the same, however. Isolated mRNAs may be utilized as templates from which purified ribosomes, or cellular extracts containing ribosomes, may “read” and translate. One of skill in the art is then able to assess whether or not translation is hindered or abolished in the presence of an exogenously added inhibitor. The mRNAs used in any of the foregoing in vitro assays may be messages that harbor only 5′ caps, only IRES elements, or both. Furthermore, such messages may be derived from eukaryotic or prokaryotic sources with modifications added to the nucleic acid, where necessary, using methods well known within the art.

Assays for virus production also provide one with the ability to determine the efficacy of such an inhibitor. Viruses that should be inhibited from infecting or replicating in a host include hepatitis C virus, hepatitis A virus, rhinovirus, poliovirus, coxsackie virus, picornavirus, hepatitis B virus, vesicular stomatitis virus, pestivirus, encephalomyocarditis virus, and plant poty virus.

Chimpanzees represent the only other species that is susceptible to HCV infection, where the infection closely resembles that seen in humans. However, there have been reports of experimental infection in tupaias, closely related to the primates, and in immunodeficient mice. Xie, Z. C. et al., Virology, 244, 513 (1998); Schinazi, R. F. et al., Antiviral Chem. Chemother. 10, 99, (1999).

Bacterial Screening Methods

A major and highly significant part of the screening methodology disclosed here involves determining the relative ability of IRES-bearing and control pro- and eukaryotic mRNAs to stimulate protein synthesis in bacterial, as well as mammalian, systems. After acquiring three appropriate mRNA types, the screening method involves standardizing the translation efficiency of each mRNA in its native system, and then conducting comparative translation assays for each in both its native and the “foreign” translation system. The screening assays involve systematic variation of kinetic parameters, temperature and mono- and divalent cation concentrations. Successful detection of IRES-based translation in bacterial systems provides an independent avenue for identifying inhibitors of such protein synthesis.

Messenger RNAs. Polycistronic mRNA from bacteriophage f1/M13 is transcribed from cloned, amplified viral RF-DNAs, while polycistronic bacteriophage f1 mRNA is extracted directly from viral particles. Capped globin mRNA is synthesized from standard cloned DNA templates. Full length HCV and other IRES-bearing mRNAs are transcribed and isolated according to standard methods well known to those skilled in the art.

Translation Systems. The standard mRNA-dependent mammalian translation system employed is the rabbit reticulocyte lysate prepared according to the original Pelham and Jackson method (See Pelham & Jackson, Eur. J. Biochem., 247-256 (1976)) and available as product L4960 from Promega Corp. The corresponding bacterial system is an mRNA-dependent E coli S30 extract, product L1030 from Promega Corp. HCV IRES-bearing and capped globin mRNAs are standardized for translation in the mammalian reticulocyte system, and optimal conditions and mRNA concentrations are ascertained in each case. Corresponding titrations for optimal translation of phage polycistronic mRNAs in bacterial extracts are routinely employed. Assays for quantitative determination of protein synthesis levels include gel electrophoresis of ³⁵S methionine-labeled proteins and subsequent quantitation of product bands by Phosporlmager analysis; and translation of mRNAs in which a luciferase reporter segment has been inserted to allow direct determination of protein synthesis in a luminometer according to standard methods.

Titrations of each mRNA in each type of cell-free extract are conducted under dual sets of optimal conditions, as determined for each mRNA at optimal temperatures (in the range of 30-37 degrees C.), mRNA concentrations and mono- and divalent cation concentrations.

Screening of Protein Synthesis Inhibitors. Initial screens are in cell-free extracts optimized for translation of the particular mRNA under study where multiple reactions are set up in standard 96-well plates. Control and treated monocistronic standard or luciferase-bearing mRNAs (see above), containing either 5′ terminal caps or IRES structures, are translated under optimal conditions in reticulocyte lysate with and without inhibitory compounds. IRES-based and bacterial mRNA translation are similarly assessed in E coli extracts.

After 30 minutes of incubation, reactions are subjected to the gel electrophoresis or luciferase assay. Initial screens utilize standard pro- and eukaryotic protein synthesis inhibitors such as antibiotics and their derivatives (see below) at a variety of concentrations, and those with significant effects are chosen for further testing. While one goal relates to inhibitors of protein synthesis initiation, compounds are also screened because of their ability to inhibit various steps of the bacterial protein synthetic pathway. Successful candidates are assayed first in vitro and then in transfected cultured liver cells.

DNA constructs for testing cap- and IRES-dependent translation in transfected liver cells are in hand, consisting of the pCM2 vector from Invitrogen, in which RNA segments are synthesized in vivo under the control of the CMV viral promoter for eukaryotic RNA polymerase II. Both mono and di-cistronic DNAs are in hand for testing, the latter encoding a CAT (chloramphenicol acetyltransferase) reporter protein under the control of the capped globin mRNA ribosome binding site, upstream from a luciferase reporter under the control of the HCV IRES. Single mRNA molecules are transcribed which stimulate translation from either or both capped and HCV-IRES translation start sites.

Successful inhibitor candidates are selected in the in vitro tests, and then further assessed in vivo in Huh-7 cultured liver cells transfected with appropriate DNAs. With cells transfected by dicistonic DNAs, the characteristic ratio of the two reporter proteins in the absence of any treatment will set a baseline for selective inhibition (defined in one embodiment as a greater than 50% decline in IRES dependent translation with no significant decline in cap-dependent protein synthesis).

II. Compounds to be screened.

The preferred compounds derived from those that have already been rigorously tested for safety in human patients because of their previous uses as anti-bacterial agents. These compounds have the advantage of entering human clinical trials earlier than would be expected for compounds not previously approved by the Food & Drug Administration that harbor the desired activity. A preferred library includes known anti-bacterial compounds, most of whose mechanisms of action are not known.

Anti-bacterial compounds that do not elicit a desired activity may be modified genetically, chemically, or biochemically. For example, unnatural amino acids may be incorporated into any peptide or compound to increase a desired activity. Examples of unnatural amino acids include, but are not limited to, dehydroaminobutyrate, lanthionine, methyl-lanthionine and dehydroalanine. Methods such as solid phase synthesis (organic synthesis or peptide synthesis) may be utilized, either to generate a new inhibitory compound or to modify an existing compound. Such methods are well described in the art.

Peptide molecules may also be inhibitory compounds. Methods for the generation of peptides to develop libraries of potential interacting compounds are well known in the art. Over the course of the last two decades these libraries have been incorporated into systems that allow the expression of random peptides on the surface of different phage or bacteria. Alternatively, such libraries may be incorporated into the two-hybrid system described above. Many publications have reported the use of phage display technology to produce and screen libraries of polypeptides of binding to a selected target. See, e.g, Cwirla et al., Proc. Natl. Acad. Sci. USA 87, 6378-6382 (1990); Devlin et al., Science 249, 404-406 (1990), Scott & Smith, Science 249, 386-388 (1990); U.S. Pat. No. 5,571,698 to Ladner et al.

Ribosomes or any subunit of the ribosome, as defined herein, may be used as the target in such an assay. For example, any ribosome-bound eukaryotic initiation factor, the 40 S subunit, the 48 S pre-initiation complex, the 60 S subunit, any of the 18 S, 5.8 S, 5 S, and 28 S rRNA molecules, or the 80 S ribosome may be used to “capture” any interacting compound, peptide, protein, or drug.

The methods used to generate the oligonucleotides encoding such a peptide library are well known within the art. For example, a oligonucleotide library may be synthesized in vitro using PCR or other amplification methods that are well known within the art. Generally, the oligonucleotide libraries comprise a unique or variable sequence region that confers diversity to the library. Diversification of the library is typically achieved by altering the coding sequence that specifies the sequence of the peptide such that a number of possible amino acids can be incorporated at certain positions. At the core of creating the library lies the construction of degenerate primers. Degenerate primers can be constructed using available automated polynucleotide synthesizers, such as one of the Nucleic Acid Synthesis Instrument Systems (Applied Biosystems).

Compounds that are useful as identified by these screens are those which do not interfere with normal mammalian translation, but do interfere with viral protein translation.

III. Methods of Use

The methods and inhibitor molecules identified by the methods may be used for the treatment or prevention of viral infections in cells, human or animal patients. The viruses that can be treated are those in which their translation is mediated by an IRES-based mechanism. Such viruses include, but are not limited to, hepatitis A virus, encephalomyocarditis virus, rhinovirus, poliovirus, coxsackie virus, other members of the picornaviridae group, and the hepatitis C virus. It is not necessary that the entire genome be translationally regulated by an IRES-based mechanism. For example, viruses such as human hepatitis B and vesicular stomatitis virus utilize IRES elements for the specific translation of reverse transcriptase and NS protein, respectively. IRES-based dependent translation has also been shown in the retrovirus family, the pestivirus family, and plant poty viruses. The dose and the range of the antiviral agent will depend on the particular agent and the type of virus being treated, as well as the route of administration. One skilled in the art, and in particular the patient's physician or pharmacist, will be able to ascertain the appropriate dose.

The antiviral compounds typically may be administered orally, intravenously, or, administered directly into cells harboring the virus. A dosage unit may comprise a single compound or mixtures thereof with other anti-viral compounds. The anti-viral compound(s) can be administered in oral dosage forms as tablets, capsules, pills, powders, granules, elixirs, tinctures, suspensions, syrups, and emulsions. The anti-viral compounds may also be administered in intravenous (bolus or infusion), intraperitoneal, subcutaneous, or intramuscular form, all using dosage forms well known to those of ordinary skill in the pharmaceutical arts.

The compounds may also be administered in an admixture with suitable pharmaceutical diluents, extenders, excipients, or carriers (collectively referred to herein as a pharmaceutically acceptable carrier or carrier materials) suitably selected with respect to the intended form of administration and as consistent with conventional pharmaceutical practices. The unit will be in a form suitable for oral, rectal, topical, intravenous injection or parenteral administration. Administration may be systemic, or in the case of localized infection, administration may be topical or local.

Effective dosages will be determined in cellular experiments and animal trials, and will center on the safe, effective dosages currently in use for typical antibiotics and other compounds already known to work by inhibiting prokaryotic-like protein synthesis. Such USP dosages are readily obtainable from the National Library of Medicine, Bethesda, Md. Examples of adult oral dosages to be used in titrating test compounds include 7.5-12.5 mg/kg erythromycin, 4 times per day; and 12.5 mg/kg of chloramphenical, 4 times per day. Examples of injected doses to be used as standards include 4-10 mg/kg of erythromycin, 4 times per day; 12.5 mg/kg of chloramphenical, 4 times per day, and 5-20 mg/kg of the aminoglycoside streptomycin, 2-4 times per day. It is likely that increased, but still safe, dosages for these compounds and their derivatives will be required as determined by these tests.

Symptoms or criteria for response to treatment center around the level of viral production in the case of HCV infection. Tests for viral circulating viral RNA levels and changes therein are standard and can be applied in cells, animals and patients. In patients, tests for liver activities, such as the ALT test, are employed. Improvement in one or more of these criteria signals an effective dosage or treatment. It is envisages that these tests, at the level of patient trials, will be conducted in a context of interferon and ribavirin usage wherein a series of standard criteria such as those above are used at regular intervals to evaluate progress.

Length of treatment would depend upon response, but would initially be at least several months in duration so as to duplicate the time-spans used for trials of interferon and small-molecule drugs such as ribavirin in the management of HCV infection.

Modifications and variations of the present invention will be obvious to those skilled in the art and are intended to come within the scope of the appended claims. The teachings of the references referred to herein are specifically incorporated herein.

We claim:

1. An antiviral composition comprising an effective amount to inhibit viral infection or replication of a compound that inhibits translation of viral mRNA by binding to a eukaryotic ribosome to prevent binding of an internal ribosome entry site (IRES) of the viral mRNA.

2. The composition of claim 1, wherein the compound sterically blocks binding of the ribosome to the IRES.

3. The composition of claim 1, wherein the compound physically blocks binding of the ribosome to the IRES.

4. The composition of claim 1, wherein the compound does not inhibit cap-dependent translation.

5. The composition of claim 1, wherein the compound inhibits binding of the eukaryotic ribosome to the IRES but does not significantly inhibit translation of capped eukaryotic mRNA.

6. The composition of claim 1, wherein the compound is selected from the group consisting of a peptide, an antibody, a synthetic organic compound, a polysaccharide and a nucleic acid molecule.

7. The composition of claim 1, wherein the molecule binds to an RNA component or a subunit of the ribosome.

8. The composition of claim 7, wherein the RNA or ribosome subunit is selected from the group consisting of 18S rRNA, 5.8S rRNA, 28S rRNA, 5S rRNA, 43S pre-initiation complex, 40S subunit, 48S pre-initiation complex, 60S subunit, and the 80S ribosome.

9. The composition of claim 8, wherein the RNA or ribosome subunit is the 40S subunit.

10. The composition of claim 1 further comprising a pharmaceutically acceptable carrier.

11. The composition of claim 1 in a dosage effective to inhibit infection or replication of a hepatitis virus.

12. The composition of claim 1 in a dosage effective to inhibit infection or replication of a picornavirus.

13. The composition of claim 1 wherein the compound is in combination with a second compound selected from the group consisting of antibiotics, antivirals, antifungals, and anti-protozoan drugs.

14. The composition of claim 1 wherein the compound is selected from the group consisting of antibiotics, antivirals, antifungals, and anti-protozoan drugs.

15. The composition of claim 14 wherein the effective dosage of the compound is higher than the effective dosage of the compound to treat bacteria or fungi.

16. A method of treating an individual in need thereof to prevent infection and/or replication of a virus comprising administering to the individual the composition of claim 1.

17. A method to screen for antiviral compounds comprising:
adding a compound to be screened for its ability to inhibit IRES-based translation by blocking one or more of the distinct sites of interaction between the eukaryotic ribosome and the IRES to a system that is capable of translating viral mRNA, wherein the viral mRNA is translated by binding of its internal ribosome entry site to a eukaryotic ribosome, and
determining if the compound inhibits translation of viral mRNA.

18. The method of claim 17 further comprising determining if capped eukaryotic mRNA is still translated.

19. The method of claim 17, wherein the compound is selected from the group consisting of a peptide, an antibody, a synthetic organic compound, a polysaccharide and a nucleic acid molecule.

20. The method of claim 18 wherein the compound is an antibiotic.

21. The method of claim 17 wherein the compound is in a library and multiple compounds from the library are screened for selective inhibition of viral mRNA translation.

22. The method of claim 17, wherein the viral RNA is from a virus selected from the group consisting of hepatitis C virus, hepatitis A virus, rhinovirus, poliovirus, coxsackie virus, picornavirus, hepatitis B virus, vesicular stomatitis virus, pestivirus, encephalomyocarditis virus, and plant poty virus.

23. The method of claim 17, wherein the translation inhibitory compound is administered to the system in combination with another anti-viral compound.

24. The method of claim 17 wherein the system is bacterial.

25. The method of claim 17 wherein the system is an animal or animal cell culture.
